(19) United States
(12) Patent Application Publication
(10) Pub. No.: US 2012/0123261 A1
(43) Pub. Date: May 17, 2012

(54) Renal Nerve Ablation Using Mild Freezing and Vibration

(76) Inventors: Mark L. Jenson, Greenfield, MN (US); Scott Smith, Chaska, MN (US)

(21) Appl. No.: 13/243,114

(22) Filed: Sep. 23, 2011

Related U.S. Application Data
(60) Provisional application No. 61/414,187, filed on Nov. 16, 2010.
(60) Provisional application No. 61/491,889, filed on May 31, 2011.

(51) Int. Cl. A61N 7/00; A61B 8/12; A61H 1/00

(52) U.S. Cl. 600/438; 601/15; 601/2

(57) ABSTRACT

A catheter includes a thermal unit provided at its distal end and configured to receive a thermal transfer fluid. The thermal unit is configured to cause formation of ice particles in perivascular renal nerve tissue adjacent the thermal unit and warm tissue of the renal artery adjacent the thermal unit to a temperature above freezing while ice particles remain formed in the perivascular renal nerve tissue. A vibration source is configured to generate vibration of the ice particles sufficient to disrupt perivascular renal nerve tissue and terminate sympathetic renal nerve activity with no or negligible damage to thawed renal artery tissue. The vibration source may be configured to generate vibration sufficient to nucleate ice formation within at least the perivascular renal nerve tissue so that ice particles form throughout the perivascular renal nerve tissue adjacent the thermal unit.

### RELATED PATENT DOCUMENTS

This application claims the benefit of Provisional Patent Application Ser. Nos. 61/414,187 filed Nov. 16, 2010 and 61/491,889 filed May 31, 2011, to which priority is claimed pursuant to 35 U.S.C. §119(e) and which are hereby incorporated herein by reference.

### SUMMARY

Embodiments of the disclosure are directed to ablating target tissue of the body, such as innervated renal tissue. Embodiments of the disclosure are directed to systems, apparatuses, and methods for ablating target tissue of the body, such as innervated renal tissue, using thermal and vibratory elements of an intravascular catheter. Embodiments of the disclosure are directed to systems, apparatuses, and methods for ablating innervated renal tissue using thermal and vibratory elements of an intravascular catheter configured to cause formation of ice particles within perivascular renal tissue and acoustically disrupt perivascular nerve tissue using vibratory energy without injuring the renal artery.

Various embodiments are directed to a catheter having a flexible shaft, a lumen arrangement extending between proximal and distal ends of the shaft, and a length preferably sufficient to access a patient's renal artery relative to a percutaneous access location. A thermal unit is provided at the distal end of the catheter and configured to cause formation of ice particles in perivascular renal nerve tissue adjacent the thermal unit and warm tissue of the renal artery adjacent the thermal unit to a temperature above freezing while ice particles remain formed in the perivascular renal nerve tissue. A vibration source is configured to generate vibration of the ice particles sufficient to disrupt perivascular renal nerve tissue and terminate sympathetic renal nerve activity with no or negligible damage to thawed renal artery tissue. The vibration source may be configured to generate vibration sufficient to nucleate ice formation within at least the perivascular renal nerve tissue so that ice particles form throughout the perivascular renal nerve tissue adjacent the thermal unit.

According to other embodiments, a catheter includes a flexible shaft having a proximal end, a distal end, a length, and a lumen arrangement extending between the proximal and distal ends. The length of the shaft is preferably sufficient to access a patient's renal artery relative to a percutaneous access location. A thermal unit is provided at the distal end of the catheter and configured to receive a thermal transfer fluid from the lumen arrangement. The thermal unit is configured to controllably freeze perivascular renal nerve tissue adjacent the renal artery and warm tissue of the renal artery to a temperature above freezing while the perivascular renal nerve tissue remains frozen. One or more temperature sensors are provided at the distal end of the catheter. A vibration source is provided at the distal end of the catheter and configured to generate vibration of the ice particles sufficient to disrupt perivascular renal nerve tissue and terminate sympathetic renal nerve activity with no or negligible damage to thawed renal artery tissue. The vibration source may be configured to generate vibration sufficient to nucleate ice formation within at least the perivascular renal nerve tissue so that ice particles form throughout the perivascular renal nerve tissue. The catheter may include a housing provided at its distal end and configured to support the thermal unit, the one or more temperature sensors, and the vibration source.

In some embodiments, a catheter includes a flexible shaft having a proximal end, a distal end, a length, and a lumen arrangement extending between the proximal and distal ends. The length of the shaft is sufficient to access a target vessel of the body relative to a percutaneous access location. A thermal unit is provided at the distal end of the catheter and configured to cause formation of ice particles in target tissue adjacent the target vessel and warm tissue of the target vessel to a temperature above freezing while ice particles remain formed in the target tissue. A vibration source is configured to generate vibration of the ice particles sufficient to disrupt the target tissue with no or negligible damage to thawed tissue of the target vessel. The vibration source may be configured to generate vibration sufficient to nucleate ice formation within the target tissue so that ice particles form throughout the target tissue. The vibration source may be disposed at the distal end of the catheter, on a separate catheter, or on an external device.

In various embodiments, methods of the disclosure involve applying cooling to tissue of a renal artery and perivascular renal nerve tissue adjacent the renal artery to lower a temperature of the perivascular renal nerve tissue to below freezing. Methods of the disclosure further involve warming the renal artery tissue to a temperature above freezing to thaw the renal artery tissue while the perivascular renal nerve tissue remains frozen, and generating vibration of the ice particles sufficient to disrupt perivascular renal nerve tissue and terminate sympathetic renal nerve activity with no or negligible damage to thawed renal artery tissue. Methods may also involve generating vibration sufficient to nucleate ice formation so that ice particles form throughout at least the perivascular renal nerve tissue.

Various method embodiments may involve ultrasonically generating vibration of the ice particles sufficient to disrupt perivascular renal nerve tissue. Such method embodiments may also involve ultrasonically generating vibration sufficient to nucleate ice formation. One or both of generating vibration of the ice particles sufficient to disrupt perivascular renal nerve tissue and generating vibration sufficient to nucleate ice formation may be performed externally of a patient. Cooling the renal artery and perivascular renal nerve tissue and warming the renal artery tissue may respectively be performed by a common thermal unit. Sensing temperature at or proximate the renal artery tissue may be performed. Some method embodiments may involve operating an intravascular ultrasound device in at least two of a nucleating ultrasound mode, a thermal ultrasound mode, and a cavitating ultrasound mode. Other method embodiments may involve operating an intravascular ultrasound device in a nucleating ultrasound mode, a thermal ultrasound mode, and a cavitating ultrasound mode. Methods may also involve measuring a depth of ice formation within at least the perivascular renal nerve tissue and a depth of the thawed tissue, such as by using an ultrasound device.

These and other features can be understood in view of the following detailed discussion and the accompanying drawings.

### BRIEF DESCRIPTION OF THE DRAWINGS

FIG. 1 is an illustration of a right kidney and renal vasculature including a renal artery branching laterally from the abdominal aorta;

FIGS. 2A and 2B illustrate sympathetic innervation of the renal artery;

FIG. 3A illustrates various tissue layers of the wall of the renal artery;

FIGS. 3B and 3C illustrate a portion of a renal nerve;

FIG. 4A shows a therapy device of an ablation catheter which includes a thermal unit and a vibration unit in accordance with various embodiments;

FIG. 4B shows deployment of the ablation catheter illustrated in FIG. 4A within a patient's renal artery in accordance with various embodiments;

FIG. 6A shows an ablation catheter deployed within a renal artery and operating in a cooling configuration in which ice particles are formed within perivascular tissue surrounding the renal artery in accordance with various embodiments;

FIG. 6B shows an ablation catheter deployed within a renal artery and operating in a warming configuration in which renal artery tissue is thawed while perivascular tissue surrounding the renal artery remains frozen in accordance with various embodiments;

FIG. 6C shows an ablation catheter deployed within a renal artery and operating in a vibratory configuration in which ice particles formed within perivascular renal tissue is disrupted so as to terminate sympathetic renal nerve activity in accordance with various embodiments;

FIG. 7 shows a therapy system configured to mildly freeze and disruptively vibrate innervated perivascular tissue surrounding a patient's renal artery; and

FIGS. 8 and 9 show different ultrasound device configurations in accordance with various embodiments.

### DETAILED DESCRIPTION

Embodiments of the disclosure are directed to apparatuses and methods for ablating target tissue of the body, such as perivascular renal nerves. Various embodiments are directed to apparatuses which include a catheter with a cooling mechanism, a warming mechanism, and a vibration mechanism. According to various embodiments, a catheter is placed in the renal artery and cools the artery and perivascular tissue surrounding the artery to a temperature at which small ice particles form in the tissue. Mild vibration, such as by ultrasound, may be used to nucleate the ice formation so that many small ice particles form throughout the tissue at a milder temperature (e.g., about −10° C.). The presence of ice nuclei increases the temperature that ice will form throughout the tissue.

The renal artery is warmed, such as by a catheter, just enough to thaw the small ice particles within the renal artery wall but not the ice particles within the perivascular tissue. A more vigorous vibration is applied, such as by ultrasonic or sonic vibration, and vibration of the ice particles breaks apart the perivascular renal nerve tissue, with no damage to the renal artery.

According to some embodiments, a catheter includes inlet and outlet lumens for heated or cooled fluid, and an array of one or more vibrating transducers. In other embodiments, a heating and cooling catheter is used with a separate vibration device. In either case, the cooling can be by convection of a cooled fluid, a phase-change cryothermal mechanism, or a Peltier effect device, for example. The same catheter can typically be used for cooling and for warming. The same device (catheter-based or external) can typically be used for mild vibration and vigorous vibration. Ultrasound or other imaging can be used to assess the depth of ice formation and depth of thawed tissue.

In accordance with further embodiments of the disclosure, a treatment apparatus includes a catheter with a cooling mechanism, a warming mechanism, and a vibration mechanism. A representative ablation methodology of the disclosure involves placing a device in the renal artery, cooling the adjacent tissues including the perivascular renal nerves to about −10° C. to form small ice particles, briefly re-warming just the artery, leaving ice particles in the perivascular renal tissue, and using ultrasound energy or other vibration to move the ice particles and mechanically break apart the perivascular nerves. Because the renal artery does not have any ice particles, the ultrasound or other vibration will have minimal effect on the renal artery.

In some embodiments, the apparatus includes a catheter with inlet and outlet lumens for heated or cooled fluid. The catheter is placed in the renal artery, cooling fluid is introduced into the catheter, and the artery and perivascular tissue are cooled to a temperature at which small ice particles form in the tissue (such as about −7° C. to about −10° C.). Mild vibration, such as by low intensity ultrasound, can be used to nucleate the ice formation so that many small ice particles form more uniformly throughout the tissue at a relatively mild freezing temperature. The tissues can be cooled slightly more than this, to ensure ice formation throughout the target tissues, or to allow time for a small amount of re-warming without melting the ice particles. The renal artery is warmed, such as by introducing warmer fluid into the same catheter or delivery of moderate intensity ultrasound, just enough to thaw the small ice particles within the artery wall. A more vigorous vibration is applied, such as by ultrasonic or sonic vibration, and vibration of the ice particles breaks apart the perivascular nerve tissue, with no damage to the artery.

According to various embodiments, an ablation apparatus includes an array of vibrating transducers which can be included in the catheter, so that cooling, mild vibration nucleation, warming, and vigorous vibration for ablation can all be performed with a single catheter device. External components can be connected to the ablation catheter to provide cooling fluid, warming fluid, energy for the vibrations, and other control or monitoring functions.

An ultrasound device implemented in accordance with various embodiments can be operated in a number of different modes. For example, an ultrasound device can be configured to selectively operate in a multiplicity of modes, including a low intensity nucleating ultrasound mode, a moderate intensity thermal ultrasound mode, and a high intensity cavitating ultrasound mode. The nucleating ultrasound mode (e.g., <<1 W/cm²) is preferably a mild ultrasound mode relative to the moderate intensity thermal ultrasound mode (e.g., ˜1 W/cm²). The cavitating ultrasound mode (e.g., >>1 W/cm²) is preferably a high intensity ultrasound (or sonic vibration) mode relative to the thermal ultrasound mode.

The nucleating ultrasound mode is preferably used to nucleate the ice formation in target tissue so that many small ice particles form more uniformly throughout the target tissue at a relatively mild freezing temperature. The thermal ultrasound mode is preferably used to warm ice formed in the renal artery while ice particles formed in the target tissue remain frozen. The cavitating ultrasound mode (or sonic vibration mode) is preferably used to vigorously disrupt the ice particles in the target tissue. It is noted that cavitation thresholds are lower at low frequencies (˜100 KPa@ 20 KHz) than at higher frequencies (>5 MPa@ several MHz) and attenuation is lower. As such, it is preferred to use low frequency cavitation pulses and high frequency heating and imaging waves. High intensity cavitation waves are preferably pulsed to avoid overheating either the tissue or the ultrasound transducer. Typically, pulse trains of a number of wavelengths are used to reduce bandwidths requirements at a duty cycle of about 1%, for example.

In some embodiments, a cryothermal type cooling catheter incorporates a thermal device at a tip region of the catheter. Warming fluid can be introduced to the thermal device using the same lumens used to transport a cryogen. In other embodiments, a heating and cooling catheter is used with a separate vibration device. The separate vibration device can be catheter-based or external. Cooling can be accomplished by convection of a cooled fluid, by a phase-change cryothermal mechanism, or by Peltier effect device, for example. The same catheter can typically be used for cooling and for warming. The same vibration device (catheter-based or external) can typically be used for mild vibration, moderate, and vigorous vibration. Ultrasound or other imaging or measuring apparatus can be used to assess the depth of ice formation and depth of thawed tissue. Thermocouples or other temperature sensors can be incorporated at the distal tip region of the catheter in accordance with various embodiments.

According to various embodiments, a method for ablating perivascular renal nerves involves cooling applied to the renal artery using an intravascular device so that the artery and perivascular tissue cool to a temperature at which small ice particles form in the tissue. Mild vibration, such as by ultrasound, is used to nucleate the ice formation so that many small ice particles form throughout the tissue at a relatively mild temperature. The arterial and perivascular tissue is briefly re-warmed to thaw the artery but not the perivascular tissue including renal nerves. Vigorous vibration is applied, such as by internal or external ultrasonic or sonic vibration, and vibration of the ice particles breaks apart the perivascular nerve tissue, with no damage to the artery. It is noted that conventional approaches to perivascular renal nerve ablation by an RF device or cryothermal device in the renal artery have had difficulty in achieving the desired nerve ablation without damaging the artery wall. Embodiments disclosed herein provide an ablation mechanism which reliably ablates innervated renal tissue but does not injure the renal artery.

Various embodiments of the disclosure are directed to apparatuses and methods for renal denervation for treating hypertension. Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal denervation can reduce blood pressure, and may be a viable treatment option for many patients with hypertension who do not respond to conventional drugs.

The kidneys are instrumental in a number of body processes, including blood filtration, regulation of fluid balance, blood pressure control, electrolyte balance, and hormone production. One primary function of the kidneys is to remove toxins, mineral salts, and water from the blood to form urine. The kidneys receive about 20-25% of cardiac output through the renal arteries that branch left and right from the abdominal aorta, entering each kidney at the concave surface of the kidneys, the renal hilum.

Blood flows into the kidneys through the renal artery and the afferent arteriole, entering the filtration portion of the kidney, the renal corpuscle. The renal corpuscle is composed of the glomerulus, a thicket of capillaries, surrounded by a fluid-filled, cup-like sac called Bowman's capsule. Solutes in the blood are filtered through the very thin capillary walls of the glomerulus due to the pressure gradient that exists between the blood in the capillaries and the fluid in the Bowman's capsule. The pressure gradient is controlled by the contraction or dilation of the arterioles. After filtration occurs, the filtered blood moves through the efferent arteriole and the peritubular capillaries, converging in the interlobular veins, and finally exiting the kidney through the renal vein.

Particles and fluid filtered from the blood move from the Bowman's capsule through a number of tubules to a collecting duct. Urine is formed in the collecting duct and then exits through the ureter and bladder. The tubules are surrounded by the peritubular capillaries (containing the filtered blood). As the filtrate moves through the tubules and toward the collecting duct, nutrients, water, and electrolytes, such as sodium and chloride, are reabsorbed into the blood.

The kidneys are innervated by the renal plexus which emanates primarily from the aorticorenal ganglion. Renal ganglia are formed by the nerves of the renal plexus as the nerves follow along the course of the renal artery and into the kidney. The renal nerves are part of the autonomic nervous system which includes sympathetic and parasympathetic components. The sympathetic nervous system is known to be the system that provides the bodies “fight or flight” response, whereas the parasympathetic nervous system provides the “rest and digest” response. Stimulation of sympathetic nerve activity triggers the sympathetic response which causes the kidneys to increase production of hormones that increase vasoconstriction and fluid retention. This process is referred to as the renin-angiotensin-aldosterone-system (RAAS) response to increased renal sympathetic nerve activity.

In response to a reduction in blood volume, the kidneys secrete renin, which stimulates the production of angiotensin. Angiotensin causes blood vessels to constrict, resulting in increased blood pressure, and also stimulates the secretion of the hormone aldosterone from the adrenal cortex. Aldosterone causes the tubules of the kidneys to increase the reabsorption of sodium and water, which increases the volume of fluid in the body and blood pressure.

Congestive heart failure (CHF) is a condition that has been linked to kidney function. CHF occurs when the heart is unable to pump blood effectively throughout the body. When blood flow drops, renal function degrades because of insufficient perfusion of the blood within the renal corpuscles. The decreased blood flow to the kidneys triggers an increase in sympathetic nervous system activity (i.e., the RAAS becomes too active) that causes the kidneys to secrete hormones that increase fluid retention and vasorestriction. Fluid retention and vasorestriction in turn increases the peripheral resistance of the circulatory system, placing an even greater load on the heart, which diminishes blood flow further. If the deterioration in cardiac and renal functioning continues, eventually the body becomes overwhelmed, and an episode of heart failure decompensation occurs, often leading to hospitalization of the patient.

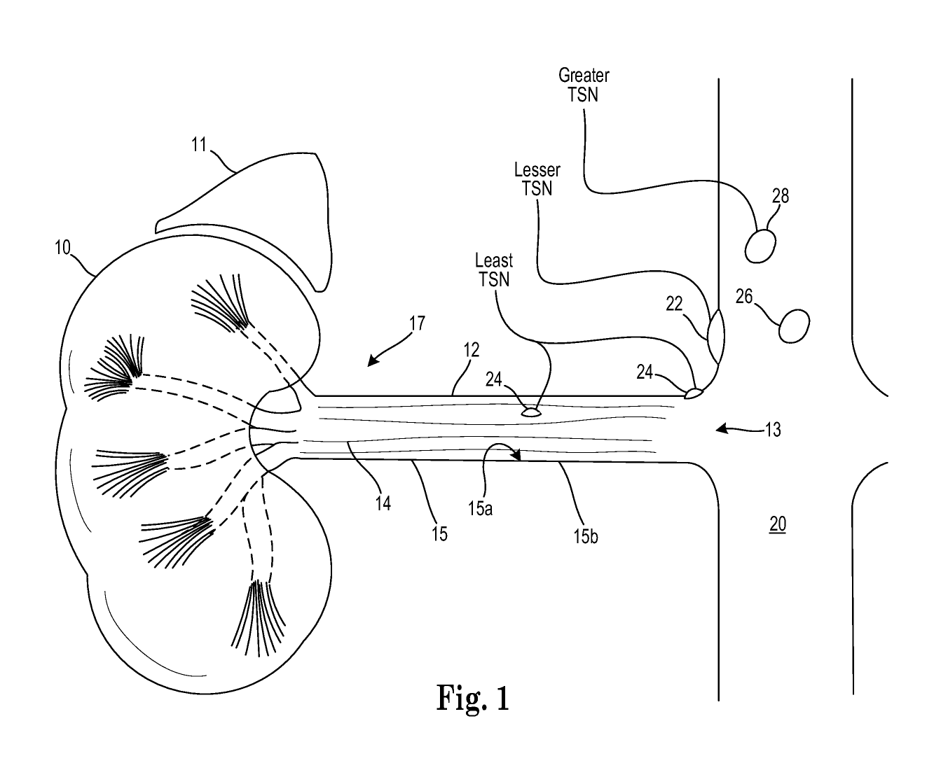

FIG. 1 is an illustration of a right kidney 10 and renal vasculature including a renal artery 12 branching laterally from the abdominal aorta 20. In FIG. 1, only the right kidney 10 is shown for purposes of simplicity of explanation, but reference will be made herein to both right and left kidneys and associated renal vasculature and nervous system structures, all of which are contemplated within the context of embodiments of the disclosure. The renal artery 12 is purposefully shown to be disproportionately larger than the right kidney 10 and abdominal aorta 20 in order to facilitate discussion of various features and embodiments of the present disclosure.

The right and left kidneys are supplied with blood from the right and left renal arteries that branch from respective right and left lateral surfaces of the abdominal aorta 20. Each of the right and left renal arteries is directed across the crus of the diaphragm, so as to form nearly a right angle with the abdominal aorta 20. The right and left renal arteries extend generally from the abdominal aorta 20 to respective renal sinuses proximate the hilum 17 of the kidneys, and branch into segmental arteries and then interlobular arteries within the kidney 10. The interlobular arteries radiate outward, penetrating the renal capsule and extending through the renal columns between the renal pyramids. Typically, the kidneys receive about 20% of total cardiac output which, for normal persons, represents about 1200 mL of blood flow through the kidneys per minute.

The primary function of the kidneys is to maintain water and electrolyte balance for the body by controlling the production and concentration of urine. In producing urine, the kidneys excrete wastes such as urea and ammonium. The kidneys also control reabsorption of glucose and amino acids, and are important in the production of hormones including vitamin D, renin and erythropoietin.

An important secondary function of the kidneys is to control metabolic homeostasis of the body. Controlling hemostatic functions include regulating electrolytes, acid-base balance, and blood pressure. For example, the kidneys are responsible for regulating blood volume and pressure by adjusting volume of water lost in the urine and releasing erythropoietin and renin, for example. The kidneys also regulate plasma ion concentrations (e.g., sodium, potassium, chloride ions, and calcium ion levels) by controlling the quantities lost in the urine and the synthesis of calcitrol. Other hemostatic functions controlled by the kidneys include stabilizing blood pH by controlling loss of hydrogen and bicarbonate ions in the urine, conserving valuable nutrients by preventing their excretion, and assisting the liver with detoxification.

Also shown in FIG. 1 is the right suprarenal gland 11, commonly referred to as the right adrenal gland. The suprarenal gland 11 is a star-shaped endocrine gland that rests on top of the kidney 10. The primary function of the suprarenal glands (left and right) is to regulate the stress response of the body through the synthesis of corticosteroids and catecholamines, including cortisol and adrenaline (epinephrine), respectively. Encompassing the kidneys 10, suprarenal glands 11, renal vessels 12, and adjacent perirenal fat is the renal fascia, e.g., Gerota's fascia, (not shown), which is a fascial pouch derived from extraperitoneal connective tissue.

The autonomic nervous system of the body controls involuntary actions of the smooth muscles in blood vessels, the digestive system, heart, and glands. The autonomic nervous system is divided into the sympathetic nervous system and the parasympathetic nervous system. In general terms, the parasympathetic nervous system prepares the body for rest by lowering heart rate, lowering blood pressure, and stimulating digestion. The sympathetic nervous system effectuates the body's fight-or-flight response by increasing heart rate, increasing blood pressure, and increasing metabolism.

In the autonomic nervous system, fibers originating from the central nervous system and extending to the various ganglia are referred to as preganglionic fibers, while those extending from the ganglia to the effector organ are referred to as postganglionic fibers. Activation of the sympathetic nervous system is effected through the release of adrenaline (epinephrine) and to a lesser extent norepinephrine from the suprarenal glands 11. This release of adrenaline is triggered by the neurotransmitter acetylcholine released from preganglionic sympathetic nerves.

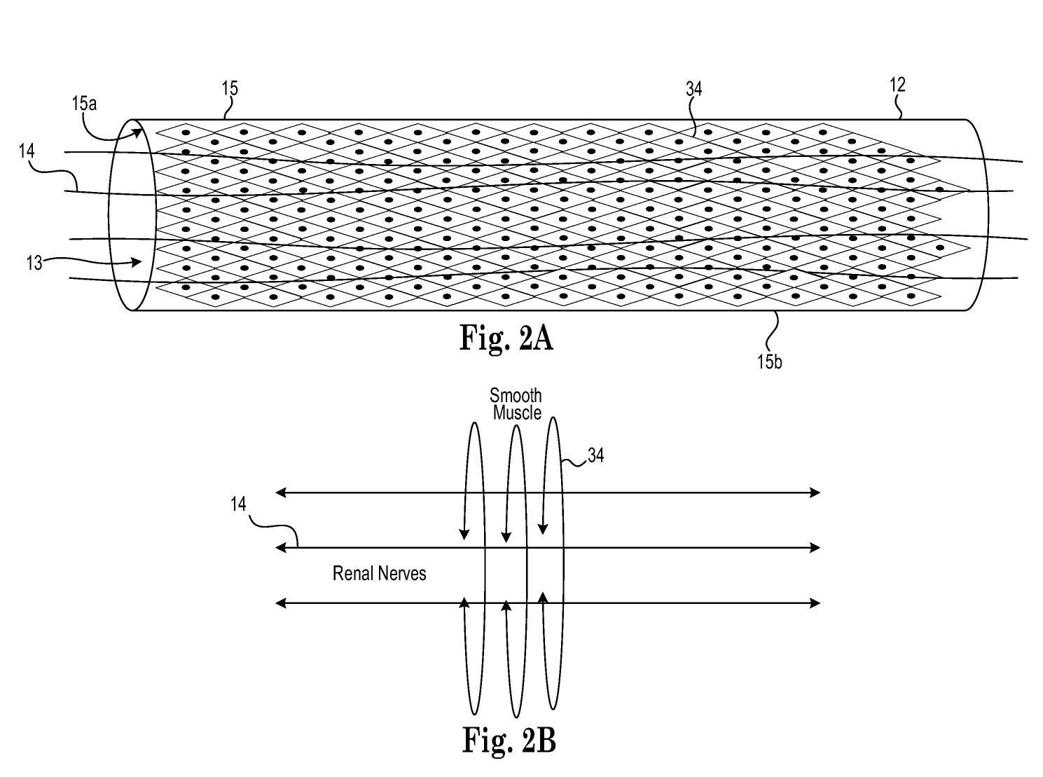

The kidneys and ureters (not shown) are innervated by the renal nerves 14. FIGS. 1 and 2A-2B illustrate sympathetic innervation of the renal vasculature, primarily innervation of the renal artery 12. The primary functions of sympathetic innervation of the renal vasculature include regulation of renal blood flow and pressure, stimulation of renin release, and direct stimulation of water and sodium ion reabsorption.

Most of the nerves innervating the renal vasculature are sympathetic postganglionic fibers arising from the superior mesenteric ganglion 26. The renal nerves 14 extend generally axially along the renal arteries 12, enter the kidneys 10 at the hilum 17, follow the branches of the renal arteries 12 within the kidney 10, and extend to individual nephrons. Other renal ganglia, such as the renal ganglia 24, superior mesenteric ganglion 26, the left and right aorticorenal ganglia 22, and celiac ganglia 28 also innervate the renal vasculature. The celiac ganglion 28 is joined by the greater thoracic splanchnic nerve (greater TSN). The aorticorenal ganglia 26 is joined by the lesser thoracic splanchnic nerve (lesser TSN) and innervates the greater part of the renal plexus.

Sympathetic signals to the kidney 10 are communicated via innervated renal vasculature that originates primarily at spinal segments T10-T12 and L1. Parasympathetic signals originate primarily at spinal segments S2-S4 and from the medulla oblongata of the lower brain. Sympathetic nerve traffic travels through the sympathetic trunk ganglia, where some may synapse, while others synapse at the aorticorenal ganglion 22 (via the lesser thoracic splanchnic nerve, i.e., lesser TSN) and the renal ganglion 24 (via the least thoracic splanchnic nerve, i.e., least TSN). The postsynaptic sympathetic signals then travel along nerves 14 of the renal artery 12 to the kidney 10. Presynaptic parasympathetic signals travel to sites near the kidney 10 before they synapse on or near the kidney 10.

With particular reference to FIG. 2A, the renal artery 12, as with most arteries and arterioles, is lined with smooth muscle 34 that controls the diameter of the renal artery lumen 13. Smooth muscle, in general, is an involuntary non-striated muscle found within the media layer of large and small arteries and veins, as well as various organs. The glomeruli of the kidneys, for example, contain a smooth muscle-like cell called the mesangial cell. Smooth muscle is fundamentally different from skeletal muscle and cardiac muscle in terms of structure, function, excitation-contraction coupling, and mechanism of contraction.

Smooth muscle cells can be stimulated to contract or relax by the autonomic nervous system, but can also react on stimuli from neighboring cells and in response to hormones and blood borne electrolytes and agents (e.g., vasodilators or vasoconstrictors). Specialized smooth muscle cells within the afferent arteriole of the juxtaglomerular apparatus of kidney 10, for example, produces renin which activates the angiotension II system.

The renal nerves 14 innervate the smooth muscle 34 of the renal artery wall 15 and extend lengthwise in a generally axial or longitudinal manner along the renal artery wall 15. The smooth muscle 34 surrounds the renal artery circumferentially, and extends lengthwise in a direction generally transverse to the longitudinal orientation of the renal nerves 14, as is depicted in FIG. 2B.

The smooth muscle 34 of the renal artery 12 is under involuntary control of the autonomic nervous system. An increase in sympathetic activity, for example, tends to contract the smooth muscle 34, which reduces the diameter of the renal artery lumen 13 and decreases blood perfusion. A decrease in sympathetic activity tends to cause the smooth muscle 34 to relax, resulting in vessel dilation and an increase in the renal artery lumen diameter and blood perfusion. Conversely, increased parasympathetic activity tends to relax the smooth muscle 34, while decreased parasympathetic activity tends to cause smooth muscle contraction.

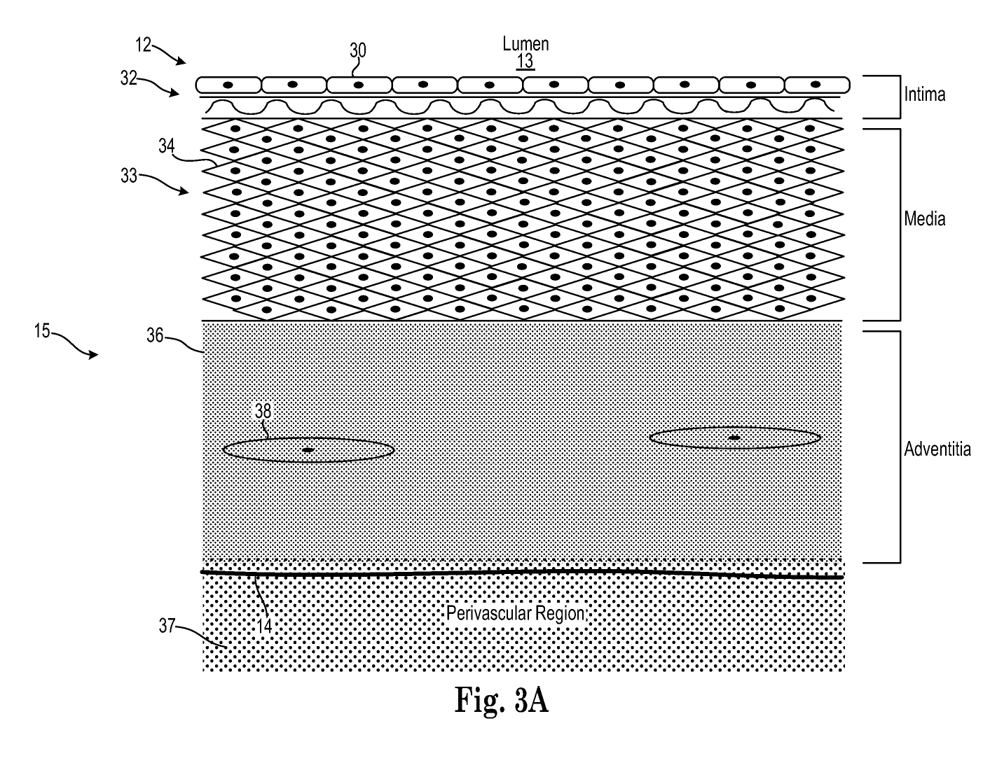

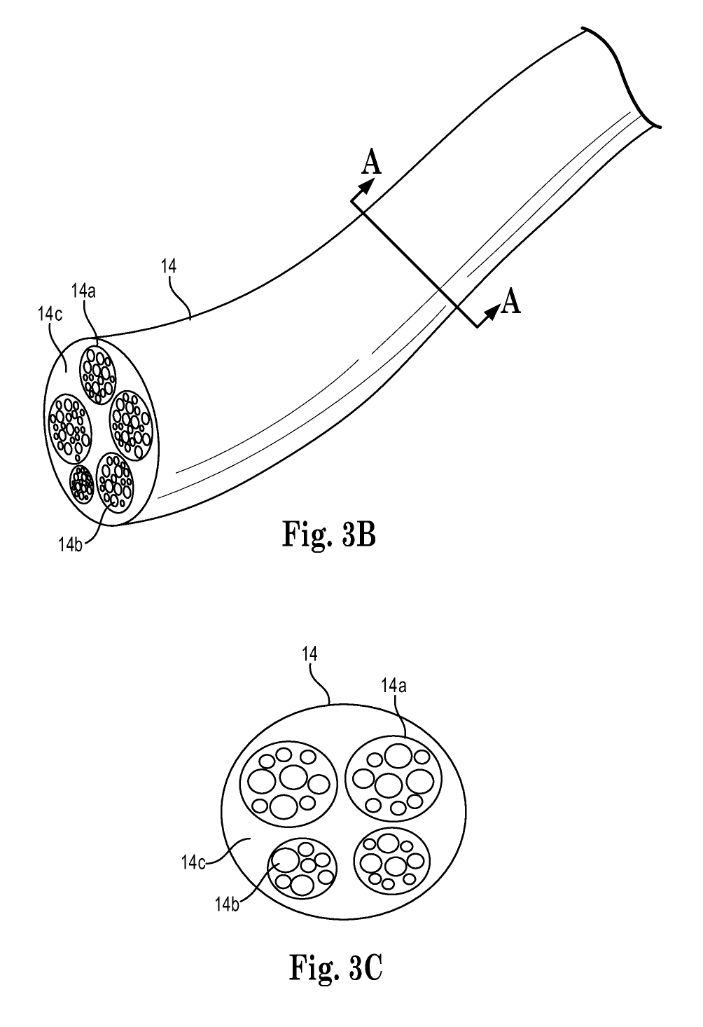

FIG. 3A shows a segment of a longitudinal cross-section through a renal artery, and illustrates various tissue layers of the wall 15 of the renal artery 12. The innermost layer of the renal artery 12 is the endothelium 30, which is the innermost layer of the intima 32 and is supported by an internal elastic membrane. The endothelium 30 is a single layer of cells that contacts the blood flowing though the vessel lumen 13. Endothelium cells are typically polygonal, oval, or fusiform, and have very distinct round or oval nuclei. Cells of the endothelium 30 are involved in several vascular functions, including control of blood pressure by way of vasoconstriction and vasodilation, blood clotting, and acting as a barrier layer between contents within the lumen 13 and surrounding tissue, such as the membrane of the intima 32 separating the intima 32 from the media 34, and the adventitia 36. The membrane or maceration of the intima 32 is a fine, transparent, colorless structure which is highly elastic, and commonly has a longitudinal corrugated pattern.

Adjacent the intima 32 is the media 33, which is the middle layer of the renal artery 12. The media is made up of smooth muscle 34 and elastic tissue. The media 33 can be readily identified by its color and by the transverse arrangement of its fibers. More particularly, the media 33 consists principally of bundles of smooth muscle fibers 34 arranged in a thin plate-like manner or lamellae and disposed circularly around the arterial wall 15. The outermost layer of the renal artery wall 15 is the adventitia 36, which is made up of connective tissue. The adventitia 36 includes fibroblast cells 38 that play an important role in wound healing.

A perivascular region 37 is shown adjacent and peripheral to the adventitia 36 of the renal artery wall 15. A renal nerve 14 is shown proximate the adventitia 36 and passing through a portion of the perivascular region 37. The renal nerve 14 is shown extending substantially longitudinally along the outer wall 15 of the renal artery 12. The main trunk of the renal nerves 14 generally lies in or on the adventitia 36 of the renal artery 12, often passing through the perivascular region 37, with certain branches coursing into the media 33 to enervate the renal artery smooth muscle 34.

Embodiments of the disclosure may be implemented to provide varying degrees of denervation therapy to innervated renal vasculature. For example, embodiments of the disclosure may provide for control of the extent and relative permanency of renal nerve impulse transmission interruption achieved by denervation therapy delivered using a treatment apparatus of the disclosure. The extent and relative permanency of renal nerve injury may be tailored to achieve a desired reduction in sympathetic nerve activity (including a partial or complete block) and to achieve a desired degree of permanency (including temporary or irreversible injury).

Returning to FIGS. 3B and 3C, the portion of the renal nerve 14 shown in FIGS. 3B and 3C includes bundles 14a of nerve fibers 14b each comprising axons or dendrites that originate or terminate on cell bodies or neurons located in ganglia or on the spinal cord, or in the brain. Supporting tissue structures 14c of the nerve 14 include the endoneurium (surrounding nerve axon fibers), perineurium (surrounds fiber groups to form a fascicle), and epineurium (binds fascicles into nerves), which serve to separate and support nerve fibers 14b and bundles 14a. In particular, the endoneurium, also referred to as the endoneurium tube or tubule, is a layer of delicate connective tissue that encloses the myelin sheath of a nerve fiber 14b within a fasciculus.

Major components of a neuron include the soma, which is the central part of the neuron that includes the nucleus, cellular extensions called dendrites, and axons, which are cable-like projections that carry nerve signals. The axon terminal contains synapses, which are specialized structures where neurotransmitter chemicals are released in order to communicate with target tissues. The axons of many neurons of the peripheral nervous system are sheathed in myelin, which is formed by a type of glial cell known as Schwann cells. The myelinating Schwann cells are wrapped around the axon, leaving the axolemma relatively uncovered at regularly spaced nodes, called nodes of Ranvier. Myelination of axons enables an especially rapid mode of electrical impulse propagation called saltation.

In some embodiments, a treatment apparatus of the disclosure may be implemented to deliver denervation therapy that causes transient and reversible injury to renal nerve fibers 14b. In other embodiments, a treatment apparatus of the disclosure may be implemented to deliver denervation therapy that causes more severe injury to renal nerve fibers 14b, which may be reversible if the therapy is terminated in a timely manner. In preferred embodiments, a treatment apparatus of the disclosure may be implemented to deliver denervation therapy that causes severe and irreversible injury to renal nerve fibers 14b, resulting in permanent cessation of renal sympathetic nerve activity. For example, a treatment apparatus may be implemented to deliver a denervation therapy that disrupts nerve fiber morphology to a degree sufficient to physically separate the endoneurium tube of the nerve fiber 14b, which can prevent regeneration and re-innervation processes.

By way of example, and in accordance with Seddon's classification as is known in the art, a treatment apparatus of the disclosure may be implemented to deliver a denervation therapy that interrupts conduction of nerve impulses along the renal nerve fibers 14b by imparting damage to the renal nerve fibers 14b consistent with neruapraxia. Neurapraxia describes nerve damage in which there is no disruption of the nerve fiber 14b or its sheath. In this case, there is an interruption in conduction of the nerve impulse down the nerve fiber, with recovery taking place within hours to months without true regeneration, as Wallerian degeneration does not occur. Wallerian degeneration refers to a process in which the part of the axon separated from the neuron's cell nucleus degenerates. This process is also known as anterograde degeneration. Neurapraxia is the mildest form of nerve injury that may be imparted to renal nerve fibers 14b by use of a treatment apparatus according to embodiments of the disclosure.

A treatment apparatus may be implemented to interrupt conduction of nerve impulses along the renal nerve fibers 14b by imparting damage to the renal nerve fibers consistent with axonotmesis. Axonotmesis involves loss of the relative continuity of the axon of a nerve fiber and its covering of myelin, but preservation of the connective tissue framework of the nerve fiber. In this case, the encapsulating support tissue 14c of the nerve fiber 14b are preserved. Because axonal continuity is lost, Wallerian degeneration occurs. Recovery from axonotmesis occurs only through regeneration of the axons, a process requiring time on the order of several weeks or months. Electrically, the nerve fiber 14b shows rapid and complete degeneration. Regeneration and re-innervation may occur as long as the endoneural tubes are intact.

A treatment apparatus may be implemented to interrupt conduction of nerve impulses along the renal nerve fibers 14b by imparting damage to the renal nerve fibers 14b consistent with neurotmesis. Neurotmesis, according to Seddon's classification, is the most serious nerve injury in the scheme. In this type of injury, both the nerve fiber 14b and the nerve sheath are disrupted. While partial recovery may occur, complete recovery is not possible. Neurotmesis involves loss of continuity of the axon and the encapsulating connective tissue 14c, resulting in a complete loss of autonomic function, in the case of renal nerve fibers 14b. If the nerve fiber 14b has been completely divided, axonal regeneration causes a neuroma to form in the proximal stump.

A more stratified classification of neurotmesis nerve damage may be found by reference to the Sunderland System as is known in the art. The Sunderland System defines five degrees of nerve damage, the first two of which correspond closely with neurapraxia and axonotmesis of Seddon's classification. The latter three Sunderland System classifications describe different levels of neurotmesis nerve damage.

The first and second degrees of nerve injury in the Sunderland system are analogous to Seddon's neurapraxia and axonotmesis, respectively. Third degree nerve injury, according to the Sunderland System, involves disruption of the endoneurium, with the epineurium and perineurium remaining intact. Recovery may range from poor to complete depending on the degree of intrafascicular fibrosis. A fourth degree nerve injury involves interruption of all neural and supporting elements, with the epineurium remaining intact. The nerve is usually enlarged. Fifth degree nerve injury involves complete transection of the nerve fiber 14b with loss of continuity.

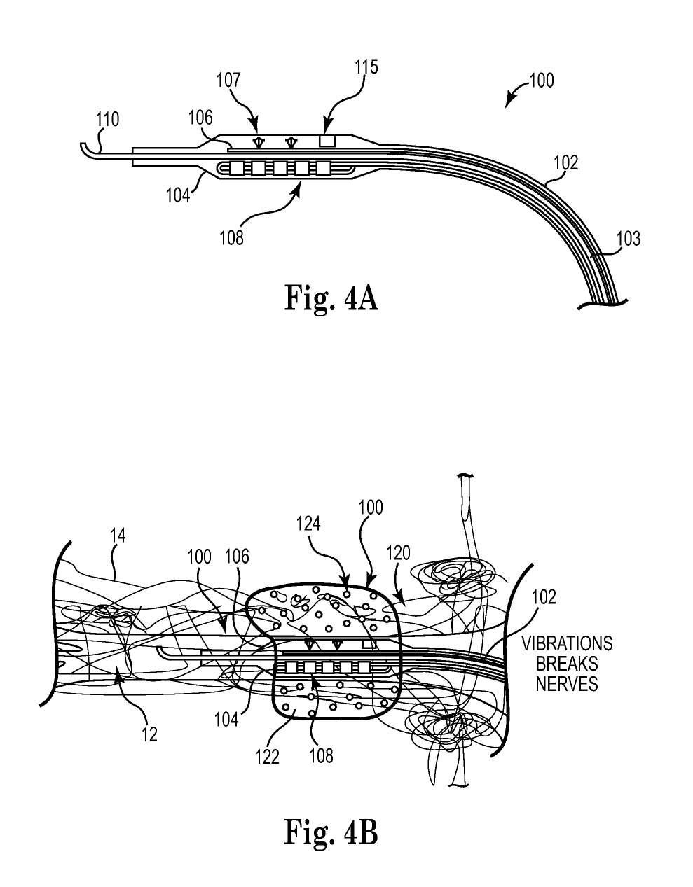

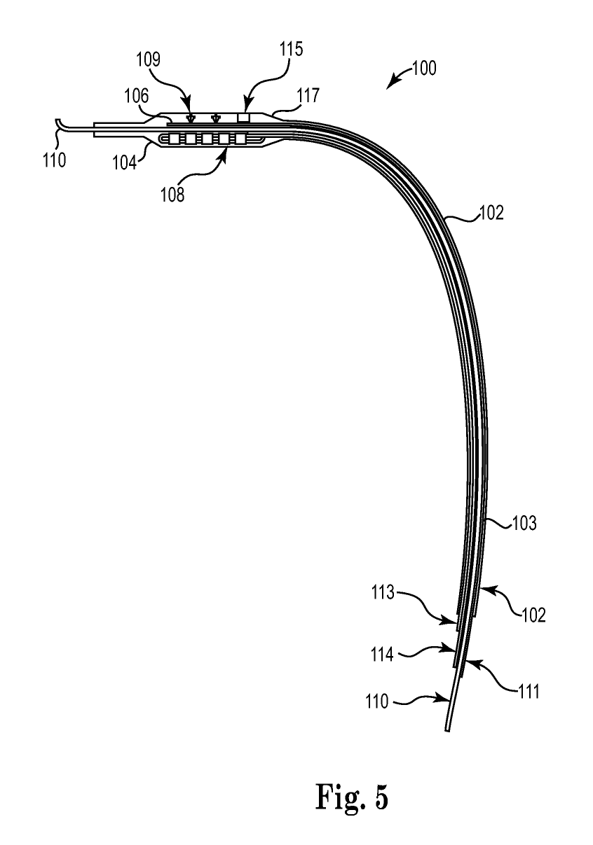

Referring now to FIG. 4A, there is shown an ablation catheter 100 configured for deployment in a renal artery of a patient. The ablation catheter 100 includes a flexible shaft 102 having a proximal end, a distal end, a length, and a lumen arrangement 103 extending between the proximal and distal ends. The length of the shaft 102 is preferably sufficient to access the patient's renal artery relative to a percutaneous access location. The ablation catheter 100 includes a therapy device 104 provided at the distal end of the shaft 102. The therapy device 104 is coupled to the lumen arrangement 103.

The therapy device 104 includes a thermal unit 106 configured to receive a thermal transfer fluid from an external source via the lumen arrangement 103. The thermal unit 106 is preferably configured to receive a cooling fluid during a freezing phase of an ablation procedure and to receive a warming fluid during a warming phase of the ablation procedure. In some embodiments, the thermal unit 106 is configured to receive a cooling fluid for use during the freezing phase, and arterial blood flowing past the therapy device 104 is used during the warming phase. In embodiments where arterial blood flow is used during the warming phase, the therapy device 104 may include a perfusion arrangement, such as fluid diversion inlet and outlet ports or channels, or a fluted housing design.

In other embodiments, the thermal unit 106 comprises one or more thermoelectric elements configured to thermally couple to the inner wall of the renal artery 12. The thermoelectric elements preferably comprise solid-state thermoelectric elements, such as Peltier elements. The thermoelectric elements can be operated in a hypothermic mode to achieve desired sub-zero temperatures in target tissue during the freezing phase. The thermoelectric elements can be operated in a hyperthermic mode to achieve desired above-zero temperatures during the warming phase. Various Peltier-effect elements and support, connection, and control arrangements and methodologies that can be adapted for use in embodiments of the present disclosure are disclosed in commonly owned U.S. Pat. No. 7,238,184 and U.S. Patent Publication Ser. No. ______, filed as U.S. patent application Ser. No. 13/157,844 on Jun. 10, 2011, which claims the benefit of U.S. Provisional Patent Application Ser. No. 61/353,853 filed Jun. 11, 2010 and entitled “RF Ablation with Stent Electrode and External Power Source,” which is incorporated herein by reference. U.S. Provisional Patent Application No. 61/353,853 filed Jun. 11, 2010, which are incorporated herein by reference.

The therapy device 104 is shown to include one or more temperature sensors 115 disposed on a housing of the therapy device 104 for sensing arterial tissue temperature. The temperature sensors 115 maybe thermocouples or other suitable temperature sensor devices. The temperature sensing response of the temperature sensors 115 is preferably sufficiently broad to sense temperatures below and above freezing.

The therapy unit 104 and the lumen arrangement 103 of the catheter shaft 102 may respectively include a lumen dimensioned to receive a guidewire 110. The guidewire 110 can be used by the clinician to access a patient's arterial system, locate the patient's renal artery 12, and advanced the ablation catheter 100 into the lumen of the renal artery 12. In some embodiments, a guide catheter may be used alone or in combination with a guidewire 110 to access the lumen of a patient's renal artery 12. Various known over-the-wire steering techniques may be used for intravascular navigation and deployment of the therapy unit 104 within the lumen of the renal artery 12.

The therapy device 104 is shown to further include a vibration source 108, such as a vibration transducer or an array of vibration transducers. The vibration source 108 may be configured to operate in one or multiple modes. In some embodiments, the vibration source 108 operates in a first vibration mode, for generating mild vibration, and a second vibration mode, for generating vigorous vibration. In other embodiments, the vibration source 108 operates in a first vibration mode, for generating mild vibration, a second vibration mode, for generating heat, and a third vibration mode for generating vigorous vibration. In further embodiments, the vibration source 108 operates in a single vibration mode for generating mild vibration. In such embodiments, a second vibration source, which can be an external or patient-internal vibration source, is used for one or both of generating heat and generating vigorous vibration.

The lumen arrangement 103 of the catheter shaft 102 includes various lumens and conductors for coupling the various components of the therapy device 104 to appropriate components of a patient-external medical system. A representative external medical system is shown in FIG. 7 and discussed in detail hereinbelow.

FIG. 4B shows an ablation catheter 100 of the type depicted in FIG. 4A deployed in a patient's renal artery 12. In the illustrative embodiment shown in FIG. 4B, a thermal transfer fluid 107 is supplied to the thermal unit 106 which cools the thermal unit 106 to a temperature below freezing. The thermal unit 106 preferably cools a region 122 of arterial and perivascular tissue 120 to a temperature at which small ice particles 124 form within this tissue. Preferably, the temperature of the thermal unit 106 is controllably lowered so that relatively mild freezing occurs in the tissue within region 122. For example, the temperature of the thermal unit 106 is controllably lowered so that the arterial and perivascular tissue 120 within region 122 is lowered to approximately −10° C. If desired, the temperature of the thermal unit 106 can be further lowered (e.g., by a few degrees, such as to a temperature of about −12° C. to about −15° C.) to ensure that ice particles have formed in the perivascular tissue 120. It is noted that nucleating temperatures for various types of tissue typically differ, and that a desired nucleating temperature in some tissues may be lethal in others. One or more temperature sensors 115 are preferably provided at the therapy device 104 for measuring arterial tissue temperature. In some embodiments, an imaging or measuring system, such as an ultrasound system, may be used to assess the depth of ice formation within the tissue of region 122.

After the tissue within region 122 is lower to the desired sub-zero temperature in which ice particles 124 form, a thermal transfer fluid 107 is supplied to the thermal unit 106 which warms tissue of the renal artery 12. Preferably, the renal artery 12 is warmed just enough to thaw the small ice particles 124 within the renal artery wall. With ice particles 124 formed in the perivascular space adjacent the renal artery 12 and the wall of the renal artery 12 thawed, vigorous vibration is applied to the frozen perivascular tissue 120 within region 122, such as by an ultrasonic cavitation or sonic vibration device. The ultrasonic cavitation or sonic vibration device may be a patient-internal device or an external device. The vibration of the ice particles 122 within the perivascular space breaks apart the perivascular nerve tissue 14, typically by cavitation or sonoporation, with no damage to the thawed tissue of the renal artery 12.

It is understood that the term “frozen” within the context of various embodiments refers to the presence of ice particles within target tissue, such as perivascular renal tissue. For example, perivascular renal tissue is considered “frozen” in the context of various embodiments when the concentration of ice particles within the cooled perivascular renal tissue provides for disruption of renal nerve fibers and termination of sympathetic renal nerve activity when the ice particles are subject to vigorous acoustic vibration.

As is shown in FIGS. 4A and 4B, the vibration unit 108 includes an array of vibration transducers, such as ultrasonic transducers. The ultrasonic transducers may include piezoelectric transducers, for example. In various embodiments, the vibration transducers of the vibration unit 108 are operable in a multiplicity of vibration modes (e.g., mild, moderate, and/or vigorous vibration modes) as discussed above. For example, the vibration unit 108 may include one or more vibration sources that are operable in at least two of mild, moderate, and vigorous vibration modes. In some configurations, one or more vibration sources are operable in mild and vigorous vibration modes, with heating provided by heated thermal transfer fluid supplied to the thermal unit 106.

By way of further example, the vibration unit 108 may include one or more vibration sources that are operable in mild, moderate, and vigorous vibration modes. The vibration unit 108 can include a nucleation vibration source and a high intensity focused ultrasound (HIFU) ultrasound source operable in a moderate thermal ultrasound mode and a high intensity cavitating ultrasound mode. As mentioned previously, the vibration unit 108 may include vibration transducers configured to operate in a mild vibration mode and an external vibration unit can be operated in one or both of the thermal and vigorous vibration modes. Accordingly, a separate fluidic thermal unit 106 need not be included in certain embodiments.

FIG. 5 shows details of a lumen arrangement 103 of an ablation catheter 100 in accordance with various embodiments. In the embodiment shown in FIG. 5, the lumen arrangement 103 includes a fluid delivery arrangement configured to transport a thermal transfer fluid to and from the distal end the thermal unit 106. The fluid delivery arrangement is fluidly coupled to a fluid source which may be configured to supply a pressurized thermal transfer fluid to the thermal unit 106.

The fluid delivery arrangement shown in FIG. 5 includes at least two lumens 111 and 113 configured as supply and return lumens for supplying a cryogen to the thermal unit 106 and returning spent cryogen or gas to the proximal end of the catheter 100, respectively. The cryogen may be circulated through the thermal unit 106 via a hydraulic circuit that includes a cryogen source, supply and return lumens 111, 113, and the thermal unit 106 of the therapy device 104 disposed at the distal end of the catheter 100. The shaft 102 of the catheter 100 is preferably lined with or otherwise incorporates insulation material(s) having appropriate thermal and mechanical characteristics suitable for a selected cryogen.

The fluid delivery arrangement is preferably fluidly coupled to a cryogen source which includes a reservoir arrangement fluidly coupled to a pump system, described hereinbelow with reference to FIG. 7. A cryogen is contained within the reservoir arrangement. A variety of cryogens may be employed, including cold saline or cold saline and ethanol mixture, Freon or other fluorocarbon refrigerants, nitrous oxide, liquid nitrogen, and liquid carbon dioxide, for example. The fluid delivery arrangement may also be configured to supply a warm thermal transfer fluid to the thermal unit 106. Preferably, warm thermal transfer fluid is communicated to and from the thermal unit 106 using the same fluid delivery arrangement that transports the cryogen to and from the thermal unit 106. Alternatively, the lumen arrangement 103 may include a separate supply and return lumen arrangement for transporting warm thermal transfer fluid to the thermal unit 106 and spent thermal fluid from the thermal unit 106.

In some embodiments, the thermal unit 106 includes a phase change cooling arrangement. According to various embodiments, a liquid cryogen is supplied to the thermal unit 106 via a supply lumen 111. When released inside the thermal unit 106, the liquid cryogen undergoes a phase change that cools the thermal unit 106 by absorbing the latent heat of vaporization from the tissue surrounding the therapy unit 104, and by cooling of the vaporized gas as it enters a region of lower pressure inside the thermal unit 106 (via the Joule-Thomson effect).

As a result of the phase change and the Joule-Thompson effect, heat is extracted from the surroundings of the therapy unit 104, thereby cooling the renal artery and perivascular tissue to a temperature below freezing. The gas released inside the thermal unit 106 is exhausted through a return lumen 113 provided in the lumen arrangement 103 of shaft 102. The pressure inside the thermal unit 106 may be controlled by regulating one or both of a rate at which cryogen is delivered and a rate at which the exhaust gas is extracted. One or more temperature sensors 115 are preferably provided at the thermal unit 106 for measuring arterial tissue temperature. The lumen arrangement 103 of the catheter shaft 102 includes one or more conductors for coupling each of the temperature sensors 115 to an external temperature unit.

In some embodiments, the lumen arrangement 103 includes a power conductor coupled to the vibration unit 108 and an external power supply. As such, electrical power is supplied to the vibration unit 108 and used to generate pressure waves having a desired frequency and amplitude. In other embodiments, the lumen arrangement 103 includes an elongated vibration element that extends between the vibration unit 108 and an external vibration generator. In such embodiments, the external vibration generator mechanically excites the elongated vibration element causing the distal end of the vibration unit 108 to vibrate at a desired frequency and amplitude. As discussed previously, the vibration unit 108 can operate in multiple modes, including a relatively mild vibration mode (to nucleate ice formation in the tissue region 122) and at a relatively vigorous vibration mode (to generate cavitation vibration within the frozen tissue region 122) sufficient to disrupt perivascular renal nerve tissue and terminate sympathetic renal nerve activity within the tissue region 122.

The therapy unit 104 includes a housing 117 which is typically constructed from polymeric material. The housing 117 preferably has a diameter dimensioned to fit within a renal artery 12 of an average patient. It is understood that different models of ablation catheters 100 can be constructed each having specific housing configurations and dimensions appropriate for a given population of patients. In some embodiments, the housing 117 may comprise an expandable element, such as a pressurizable balloon or a mechanically expandable arrangement. Use of such an expandable element in the construction of the housing 117 allows for use of a common housing design for a population of patients having varying anatomy. In accordance with various embodiments in which a pressurizable balloon is used in the construction of the housing 117, a thermal transfer fluid may be used for pressurizing the balloon and one or both of cooling and warming renal artery tissue and innervated perivascular tissue. A separate pressurizable lumen arrangement can be provided within the catheter shaft 102 for controlling the pressure within the balloon.

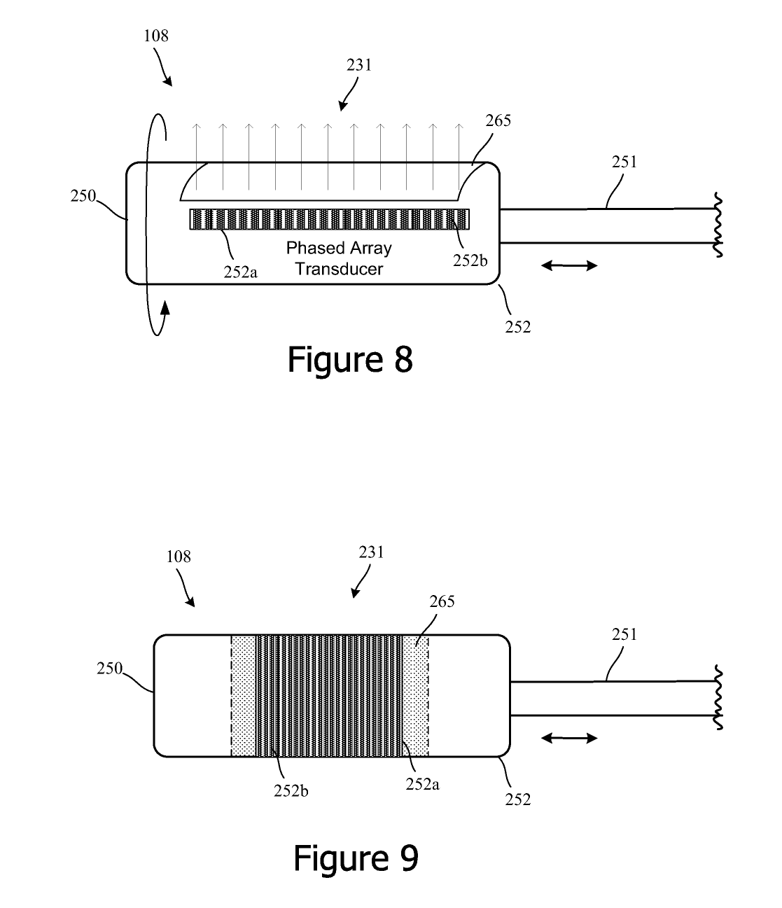

FIGS. 8 and 9 show vibration units 108 implemented in accordance with various embodiments. In the embodiments shown in FIGS. 12 and 13, the vibration units 108 each include an ultrasound unit 250. An emitter 252 of the ultrasound unit 250 includes an acoustic phased array transducer 252a which comprises a multiplicity of acoustic elements 252b. The phased array transducer 252a shown in FIG. 8 extends over a radial segment of the ultrasound unit's circumference, allowing an acoustic energy beam 262 to pass through an aperture 265 (e.g., focusing lens arrangement) and impinge on target tissue. The emitter 252 of the ultrasound unit 250 may be aimed at target tissue by rotating and translating the catheter 251 (i.e., the body of the medical device) or by moving the ultrasound unit 250 relative to the catheter 251, either manually or robotically.

In the embodiment shown in FIG. 13, a phased array transducer 252a extends over all or nearly all of the ultrasound unit's circumference, allowing an acoustic energy beam 262 to pass through an annular aperture 265 (e.g., focusing lens arrangement) and impinge on a circular or cylindrical target tissue region. This embodiment of ultrasound unit 250 is particularly useful when treating an entire circumferential region 122 of the renal artery and perivascular tissue without having to translate or rotate the catheter 102 or ultrasound unit 250.

The ultrasound unit 250 preferably has a capability that allows for focusing of acoustic energy at desired distances so that all or most of the perivascular space adjacent the outer wall of the renal artery can be subject to one or both of mild and vigorous vibration. Details of these and other ultrasound apparatuses and methods are described in commonly owned U.S. Patent Publication Ser. No. ______, filed as U.S. patent application Ser. No. 13/086,116 on Apr. 13, 2011, which claims the benefit of U.S. Provisional Patent Application Ser. No. 61/324,164 filed Apr. 14, 2010 and entitled “Focused Ultrasound for Renal Denervation,” which is incorporated herein by reference.

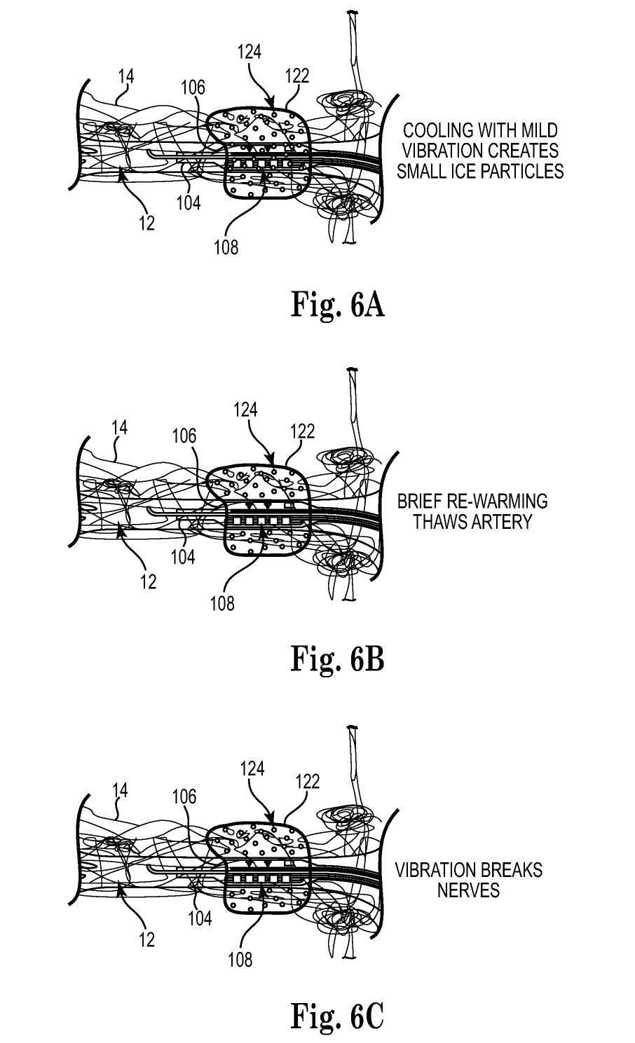

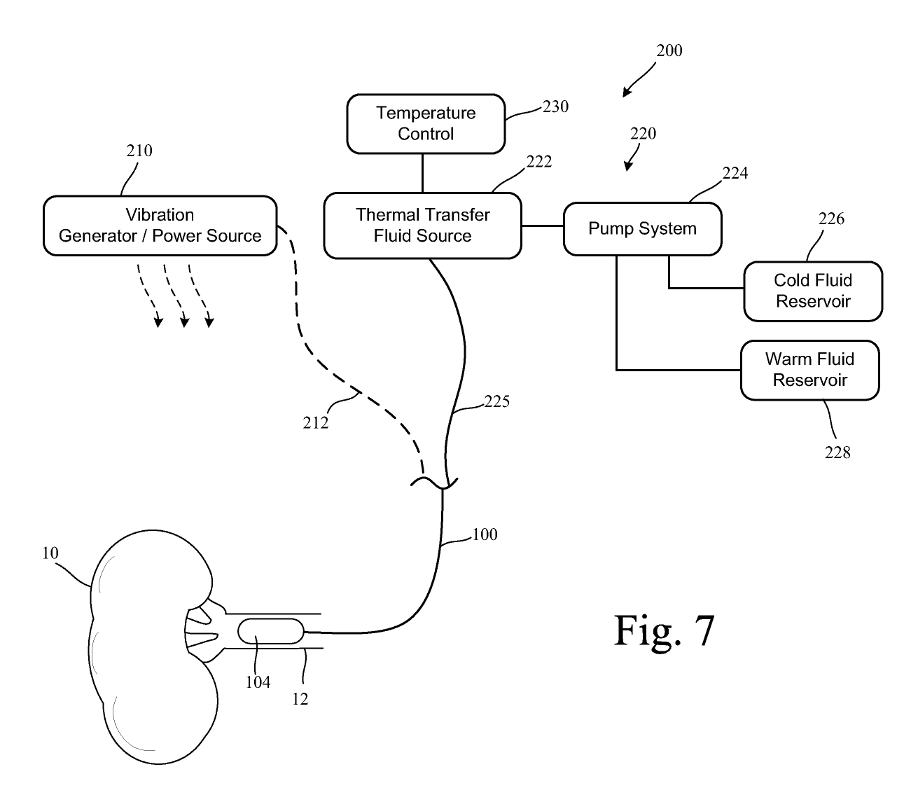

FIGS. 6A through 6C illustrate various processes of a renal ablation methodology in accordance with various embodiments. The reference numbers shown in FIGS. 6A-6C refer to elements with corresponding reference numbers discussed previously with regard to FIGS. 4A through 5. In FIG. 6A, the thermal unit 106 of the therapy device 104 is cooled to a temperature below freezing, such as between about −7° C. and about −10° C. Optionally, during the cooling procedure, the vibration unit 108 is controlled to generate mild vibration sufficient to nucleate the ice formation within a tissue region 122 surrounding the therapy device 104. The degree of mild vibration is selected to nucleate the ice formation within region 122 so that many small ice particles form throughout the tissue region 122 at relatively mild freezing temperatures.

In some embodiments, one or more temperature sensors 115 can be used to measure the arterial tissue temperature and confirm that the tissue temperature is within the desired mild freezing temperature range. In such embodiments, cooling can continue for a duration of time sufficient to freeze tissue within the perivascular space adjacent the renal artery. This duration of time can be determined from clinical experimentation, for example. In other embodiments, the vibration unit 108 can be operated in an imaging mode, allowing for in-situ measurement of the depth of ice formation within the perivascular tissue. Alternatively, an external imaging system can be used for ice depth assessment.

FIG. 6B is intended to show warming of renal artery wall tissue using warm thermal transfer fluid supplied to the thermal unit 106 during a warming phase. The warming phase is preferably sufficient in duration to allow thawing of renal artery wall tissue while the perivascular tissue remains frozen. In some embodiments, thawing of the renal artery wall tissue can be assessed using one or more temperature sensors 115 provided at the therapy device 104. The duration of the warming phase can be determined from clinical experimentation. In other embodiments, thawing of the renal artery wall tissue can be assessed using the vibration unit 108 operated in an imaging mode or by an external imaging system, each of which can provide depth measurements of thawed renal artery tissue.

FIG. 6C is intended to show vibration of ice particles 124 within frozen perivascular tissue sufficient to break apart perivascular renal tissue. As previously described, ultrasonic cavitation or sonic vibration produced by the vibration unit 108 preferably operates to disrupt ice particles and renal nerve tissue within the frozen perivascular tissue while the thawed renal artery tissue remains uninjured.

Referring now to FIG. 7, there is shown a system 200 for ablating tissue that influences sympathetic renal nerve activity in accordance with various embodiments. The system 200 shown in FIG. 7 includes a therapy device 104 provided at the distal end of an ablation catheter 100 deployed within a patient's renal artery 12. The therapy device 104 includes a thermal unit and a vibration unit of a type previously described. The ablation catheter 100 is fluidly coupled to a thermal transfer fluid source 222.

As discussed previously, mild, moderate, and vigorous vibration waves can be generated using a variety of vibration source configurations. For example, in some embodiments, the vibration unit 108 of the ablation catheter 100 is electrically coupled to an external vibration generator/power source 210. In other embodiments, the ablation catheter 100 includes an elongated vibration element which is mechanically coupled to the external vibration generator/power source 210. In further embodiments, the vibration generator/power source 210 is configured to generate mild and vigorous vibration externally of the patient. In such embodiments, the vibration generator/power source 210 may include one or more ultrasonic devices (e.g., a lithotripsy system), in which case the therapy device 104 does not include a vibration unit 108 but includes the thermal unit 106.

The thermal transfer fluid source 222 is shown coupled to a temperature control 230. The temperature control 230 is preferably coupled to one or more temperature sensors 115 provided at the therapy device 104. The temperature control 230 generates temperature signals which are used by the thermal transfer fluid source 222 to adjust (automatically via a processor of the system 200 or semi-automatically) the transport of a thermal transfer fluid to and from the thermal unit 106 for achieving desired temperatures within the renal artery and perivascular tissue surrounding the therapy device 104.

A pump system 224 is shown coupled to the thermal transfer fluid source 222. The pump system 224 is coupled to a fluid reservoir system which can include a cold fluid reservoir 226 and a warm fluid reservoir 228. The cold and warm fluid reservoirs 226 and 228 can be selectively fluidly coupled to the ablation catheter 100 via a fluid coupling arrangement 225 in accordance with the various phases of an ablation procedure. A variety of cryogens may be stored in the cold and warm fluid reservoirs 226 and 228. Suitable thermal transfer fluids for use in the thermal transfer fluid arrangement shown in FIG. 7 include cold saline or cold saline and ethanol mixture, Freon or other fluorocarbon refrigerants, nitrous oxide, liquid nitrogen, and liquid carbon dioxide, for example.

Various embodiments disclosed herein are generally described in the context of ablation of perivascular renal nerves for control of hypertension. It is understood, however, that embodiments of the disclosure have applicability in other contexts, such as performing ablation from within other vessels of the body, including other arteries, veins, and vasculature (e.g., cardiac and urinary vasculature and vessels), and other tissues of the body, including various organs.

It is to be understood that even though numerous characteristics of various embodiments have been set forth in the foregoing description, together with details of the structure and function of various embodiments, this detailed description is illustrative only, and changes may be made in detail, especially in matters of structure and arrangements of parts illustrated by the various embodiments to the full extent indicated by the broad general meaning of the terms in which the appended claims are expressed.

What is claimed is:

1. An apparatus, comprising:
a catheter comprising a flexible shaft having a proximal end, a distal end, a length, and a lumen arrangement extending between the proximal and distal ends, the length of the shaft sufficient to access a patient's renal artery relative to a percutaneous access location;
a thermal unit provided at the distal end of the catheter and configured to cause formation of ice particles in perivascular renal nerve tissue and warm tissue of the renal artery to a temperature above freezing while ice particles remain formed in the perivascular renal nerve tissue; and
a vibration source configured to generate vibration of the ice particles sufficient to disrupt perivascular renal nerve tissue and terminate sympathetic renal nerve activity with no or negligible damage to thawed renal artery tissue.

2. The apparatus of claim 1, wherein the vibration source is configured to generate vibration sufficient to nucleate ice formation within at least the perivascular renal nerve tissue.

3. The apparatus of claim 1, wherein the vibration source is provided at the distal end of the catheter.

4. The apparatus of claim 1, wherein the vibration source is provided at the distal end of a second catheter having a length sufficient to access a body location in or near the patient's renal artery.

5. The apparatus of claim 1, wherein the vibration source comprises an external vibration source.

6. The apparatus of claim 1, wherein the vibration source comprises an ultrasound source.

7. The apparatus of claim 1, wherein the vibration source is configured to generate acoustic energy sufficient to disrupt perivascular renal nerve tissue by way of a cavitation mechanism or a sonoporation mechanism.

8. The apparatus of claim 1, wherein:
the thermal unit comprises an ultrasound device; and
the vibration source comprises the ultrasound device or a separate ultrasound device.

9. The apparatus of claim 1, wherein one of the thermal unit and the vibration source comprises an ultrasound device configured to measure a depth of ice formation within at least the perivascular renal nerve tissue and a depth of the thawed tissue.

10. The apparatus of claim 1, wherein the thermal unit and the vibration source comprise a common ultrasound device selectively operable in at least two of a mild nucleating ultrasound mode, a thermal ultrasound mode, and a cavitating ultrasound mode.

11. The apparatus of claim 1, wherein the thermal unit is configured to provide cooling by at least one of convection of a cooled fluid, a phase-change cryothermal mechanism, and a Peltier effect.

12. The apparatus of claim 1, comprising one or more temperature sensors provided at the distal end of the catheter.

13. The apparatus of claim 1, wherein the thermal unit comprises:
a first arrangement configured to receive a thermal transfer fluid for controllably freezing the perivascular renal nerve tissue; and
a second arrangement configured to receive a thermal transfer fluid for controllably warming the arterial tissue.

14. The apparatus of claim 1, wherein the lumen arrangement comprises a guide lumen dimensioned to receive a guidewire.

15. An apparatus, comprising:
a catheter comprising a flexible shaft having a proximal end, a distal end, a length, and a lumen arrangement extending between the proximal and distal ends, the length of the shaft sufficient to access a target vessel of the body relative to a percutaneous access location;
a thermal unit provided at the distal end of the catheter and configured to cause formation of ice particles in target tissue adjacent the target vessel and warm tissue of the target vessel to a temperature above freezing while ice particles remain formed in the target tissue; and
a vibration source configured to generate vibration of the ice particles sufficient to disrupt the target tissue with no or negligible damage to thawed tissue of the target vessel.

16. The apparatus of claim 15, wherein the vibration source is configured to generate vibration sufficient to nucleate ice formation within the target tissue.

17. The apparatus of claim 15, wherein the thermal unit and the vibration source comprise a common ultrasound device or separate ultrasound devices selectively operable in at least two of a mild nucleating ultrasound mode, a thermal ultrasound mode, and a cavitating ultrasound mode.

18. The apparatus of claim 15, wherein one of the thermal unit and the vibration source comprises an ultrasound device configured to measure a depth of ice formation within at least the target tissue and a depth of the thawed target vessel tissue.

19. A method, comprising:
applying cooling to tissue of a renal artery and perivascular renal nerve tissue adjacent the renal artery to lower a temperature of the perivascular renal nerve tissue to below freezing;
warming the renal artery tissue to a temperature above freezing to thaw the renal artery tissue while the perivascular renal nerve tissue remains frozen; and
generating vibration of the ice particles sufficient to disrupt perivascular renal nerve tissue and terminate sympathetic renal nerve activity with no or negligible damage to thawed renal artery tissue.

20. The method of claim 19, further comprising generating vibration sufficient to nucleate ice formation so that ice particles form throughout at least the perivascular renal nerve tissue.

21. The method of claim 19, wherein generating vibration of the ice particles comprises ultrasonically generating cavitating vibration of the ice particles sufficient to disrupt perivascular renal nerve tissue and terminate sympathetic renal nerve activity.

22. The method of claim 19, wherein:
cooling the renal artery and perivascular renal nerve tissue is performed by a first thermal unit; and
warming the renal artery tissue is performed by an ultrasound device.

23. The method of claim 19, wherein:
warming the renal artery tissue is performed by an ultrasound device operating in a thermal ultrasound mode; and
generating vibration of the ice particles sufficient to disrupt perivascular renal nerve tissue and terminate sympathetic renal nerve activity is performed by the ultrasound device or a separate ultrasound device operating in a cavitating ultrasound mode.

24. The method of claim 19, comprising measuring a depth of ice formation within at least the perivascular renal nerve tissue and a depth of the thawed tissue.
